(19) United States
(12) Patent Application Publication
(10) Pub. No.: US 2014/0287517 A1
(43) Pub. Date: Sep. 25, 2014

(54) SK

(71) Applicant: Alfred M. Haas, Oakhurst, NJ (US)

(72) Inventor: Alfred M. Haas, Oakhurst, NJ (US)

(21) Appl. No.: 14/214,575

(22) Filed: Mar. 14, 2014

Related U.S. Application Data
(60) Provisional application No. 61/785,103, filed on Mar. 14, 2013.

(51) Int. Cl. G01N 33/18

(52) U.S. Cl. CPC G01N 33/18
USPC 436/124; 422/560; 436/163

(57) ABSTRACT

This invention relates to methods and means for performing pool and other water and liquid testing. Some embodiments of the methods and means of the invention also incorporate additional functionality including, but not limited to communication, sensing, display and data processing elements. Various embodiments of the methods and means of the invention may be performed by and/or implemented in hardware, in software, by one or more entities, and/or by some combination of hardware, software and/or one or more entities.

### CROSS-REFERENCE TO RELATED APPLICATIONS

Pursuant to 35 USC §119(e) and as set forth in the Application Data Sheet, this utility application claims the benefit of priority from U.S. Provisional Patent Application No. U.S. 61/785,103 (“the '103 provisional”) which is incorporated herein in its entirety by reference.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

This invention claims priority from the '103 provisional and expressly incorporates by reference the disclosures contained therein in their entirety, including but not limited to all patents, patent applications, and publications which are incorporated by reference in the '103 provisional and which are incorporated by reference herein in their entirety.

In the context of the instant disclosure, the term “software” is taken in the broadest sense of its ordinary meaning and illustrative examples may comprise, but are not limited to, one or more of the following: realized embodiments of algorithms in any form, code written in whole or in part in any programming, scripting or other language (including, but not limited to, popular languages such as C++, Java, Visual Basic, Python, PHP, HTML, and/or device specific machine or assembly languages, etc. . . . ), programs, mobile and/or other applications (e.g., those for Android and iOS based systems), applets, scripts, operating systems (OS) and components of OS, embedded and other software and instructions, structured data, op codes, commands, executables, firmware, drivers, virtual machines, and/or instruction sets for a system. Software may operate at many levels including, but not limited to, over a distributed system (e.g., on a cloud computing or mobile network), on a particular device, on a local computer or other machine, embedded in an ASIC or other circuit, and running on top of one or more real or virtual levels, including but not limited to an OS and a hardware level.

In the context of the instant disclosure, the term “hardware” is taken in the broadest sense of its ordinary meaning and illustrative examples may comprise, but are not limited to, one or more of the following: computers, smart phones, PDAs, other commercially available electronic devices such as tablet PCs, netbooks, e-readers (e.g. Kindle™ and Nook™), pagers, alarms, beepers, cell phones, hearing aids, watches comprising integrated and/or discrete circuits, monitors and displays, televisions, calculators, iPods™ and MP3 players, radios and stereos, remote controls, bar code readers, keyboards, cameras, other input devices, data acquisition systems, other physical devices and systems comprising integrated and/or discrete circuits, CPUs, hard drives, flash USB drives, other flash and solid state drives, programmable logic arrays, FPGAs, CPLDs, microcontrollers, DSPs, receivers, transmitters, drivers, ADC's (analog-to-digital converters), DAC's (digital-to-analog converters), decoders, multiplexers, comparators, latches, gates, op amps, LNA (low noise amplifiers), PLL (phase locked loops), antennae, radio frequency identification (“RFID”) devices, near-field communication (“NFC”) devices, coils, capacitors, inductors, resistors, transformers, solenoids, other analog circuits and components, other digital circuits and components, other mixed-signal circuits and components, optical circuits, other electromagnetic circuits and components, biological and/or chemical circuits, assemblies of memristors, carbon nanotubes, and other circuits and systems comprised of circuits.

Some embodiments of the methods and means of the instant invention may employ one or more existing wireless and/or wired communication protocols, or other custom protocols. Illustrative examples of current and historical protocols, programs and standards for digital communication include: the Internet Protocol Suite; e-mail protocols such as POP (Post Office Protocol), SMTP (Simple Mail Transfer Protocol), IMAP (Internet Message Access Protocol), and MAPI (Messaging Application Programming Interface); web browsers such as Safari™, Internet Explorer™ and Firefox™; messaging programs, protocols and standards such as WLM (Windows Live Messenger), MSNP (Microsoft Notification Protocol), AIM (AOL Instant Messenger), ICQ, XMPP (Extensible Messaging and Presence Protocol), IRC (Internet Relay Chat), MIM (Mobile Instant Messaging), SMS (Short Message Service), WAP (Wireless Area Protocol), GPRS (General Packet Radio Service), WLAN (Wireless Local Area Network), Bluetooth™, and Skype™; mobile standards such as GSM (Global System for Wideband Communications), W-CDMA (Wideband Code Division Multiple Access), LTE (Long Term Evolution), and LTE-Advanced, WirelessMAN (Metropolitan Area Networks)-Advanced; NFC (near-field communications), and many others not addressed here. To the extent that documented versions of these protocols, programs and standards are publicly accessible they are incorporated herein by reference. Likewise, some embodiments of the methods and/or means of the instant invention may employ analog and/or mixed-signal methods of communicating data or information. In addition, some embodiments of the invention employ GPS (Global Positioning System) and aGPS (Assisted Global Positioning System) protocols and/or standards.

The following publications contain information related to the design, construction, development, fabrication, production, assembly, and other aspects of some embodiments of the disclosed invention—including, but not limited to, wearable and other devices, software and hardware such as sensors and transducers, transmitters, receivers and other circuits, housings, optics. programmable logic elements and chips, custom ASICs, electrical and mechanical switches, electrical and mechanical regulators, etc.: Analysis and Design of Analog Integrated Circuits by Paul R. Gray, Paul J. Hurst, Stephen H. Lewis, Robert G. Meyer, published by John Wiley & Sons, copyright 2001; Digital Principles and Design by Donald D. Givone, published by McGraw Hill copyright 2003; Physics by Paul A. Tipler, published by Worth Publishers, copyright 1976; The New Way Things Work by David Macaulay, published by Houghton Mifflin, copyright 1988; CMOS Circuit Design, Layout and Simulation by R. Jacob Baker, published by the Institute of Electrical and Electronics Engineers, copyright 2005; Microelectronic Circuits by Adel S. Sedra and Kenneth C. Smith, published by Oxford University Press, copyright 1998; Thin Film Technology Handbook by Aicha Elshabini-Riad, Fred D. Barlow III, published by McGraw-Hill, copyright 1998; Field and Wave Electromagnetics by David K. Cheng, published by Addison-Wesley, copyright 1989; VLSI for Wireless Communications by Bosco Leung, published by Prentice Hall, copyright 2002; Complete Wireless Design by Cotter W. Sayre, published by McGraw Hill, copyright 2001; Pattern Classification, Second edition by Richard Duda, Peter Hart and David Stork, published by John Wiley & Sons, Inc., copyright 2001; C++ How to Program, Third edition by H. Dietel & P. Dietel, published by Prentice Hall, copyright 2001; Professional Android 2 Application Development by Roto Meier, published by Wiley Publishing, Inc., copyright 2010; the various versions of the Android SDK; the various versions of the Internet Protocol Suite; the various versions of the iOS SDK; the various versions of the Windows and Windows Mobile SDKs. All publications cited herein are hereby incorporated by reference in their entirety.

The discussion of the background of the invention herein is included to explain the context of the invention. Although each of the patents, patent applications, and publications cited herein are hereby incorporated by reference, neither the discussion of the background nor the incorporation by reference is to be taken as an admission that any aspect, element, embodiment, or feature of the invention was published, known, or part of the common general knowledge as of the priority date of any claims of the invention.

### BRIEF SUMMARY OF THE INVENTION

The instant invention relates to methods and means for performing pool and other water and liquid testing.

### BRIEF DESCRIPTION OF THE SEVERAL VIEWS OF THE DRAWINGS

FIG. 1 illustrates one specific example of an embodiment of the instant invention comprising a two-pronged clip securing a test strip to a handle.

FIG. 2 illustrates another specific example of an embodiment of the instant invention comprising a handle with a roll of test strips inside a portion of the handle, one of said test strips being exposed through a slit in an end of said handle.

FIG. 3 illustrates one specific example of an embodiment of the instant invention comprising an electric test device embedded in an end of a handle; in this specific example, the OLED screen displays salient data and the green light indicates water quality is within desired parameters.

FIGS. 4-11 are 3D schematics of several specific examples of embodiments of the instant invention comprising clips. FIGS. 4-7 show different views of an example of an embodiment of a clip that may be detachably affixed to a handle and which is used to secure a test strip as shown in FIG. 18.

FIGS. 8-11 illustrate different views of an example of an embodiment of a clip that may be detachably affixed to a handle and which may be used to secure a test strip by, e.g., inserting one end of the strip through one slot and bending it back to insert the opposite direction through the other slot.

FIGS. 12 A-C illustrate specific examples of embodiments of the instant invention comprising bands—respectively elastic, tied, and screw-tied.

FIGS. 13 A-C illustrate specific examples of embodiments of the instant invention comprising pins—respectively straight, screw-pin, and through-hole-pin.

FIG. 14 is a photograph of a bottle of one brand of chemical test strips and a single unused test strip alongside it.

FIG. 15 is a photograph of a specific example of an embodiment of a clip alongside the tip of a handle and a test strip.

FIG. 16 are a pair of photographs of a specific example of an embodiment of a clip affixed to a handle.

FIG. 18 is a photograph of a specific example of an embodiment of a clip affixed to a handle with a test strip secured by the clip.

### DETAILED DESCRIPTION OF THE INVENTION

This invention relates to methods and means for performing pool and other water and liquid testing. Some embodiments of the methods and means of the invention also incorporate additional functionality including, but not limited to communication, sensing, display and data processing elements.

In some embodiments of the invention, the testing method comprises attaching a chemical test strip to a handle having a means for securing the test strip to the handle, then submersing all or a portion of the handle and the test strip into a region of a liquid for some period of time, and then removing all or a portion of the handle and test strip from the liquid for evaluation. In some embodiments of the invention, a method for testing comprises completely and/or partially dispensing one or more chemical test strips from a handle comprising one or more chemical test strips, then submersing all or a portion of the handle and test strip into a region of a liquid for some period of time, and then removing all or a portion of the handle and test strip for evaluation.

In some embodiments a method for testing comprises submersing all or a portion of a handle comprising test strips into a region of a liquid then completely and/or partially exposing one or more regions of one or more chemical test strips to the liquid for some period of time, and then removing all or a portion of the handle and test strip for evaluation.

In some embodiments, an unwrappable (or otherwise exposable) watertight roll of test strips may be affixed to a portion of a handle and individual test strips may be unrolled, peeled off, or otherwise exposed to permit testing. In some embodiments a handle may be printed with a color legend or other markings to aid in evaluating chemical test strips or the evaluation of data from other devices or apparatuses.

In some embodiments, testing comprises submersing all or a portion of an electronic device for evaluating liquid characteristics which may include but are not limited to pH, free chlorine, total chlorine, and alkalinity—said device being affixed to and/or comprising a handle—into a region of a liquid for some period of time and then removing all or a portion of the device for evaluation.

In some embodiments testing comprises submersing all or a portion of a test vial affixed and/or attached to a handle into a region of a liquid for some period of time and then removing all or a portion of the vial and liquid contained therein for evaluation.

In some embodiments, means for securing a chemical test strip or other test device to a handle may be integrated with or a part of the handle; in some embodiments, means for securing a chemical test strip or other test device to the handle may be detachable and/or removable.

In some embodiments, a handle may be telescoping; in some embodiments a handle may be of fixed length. In some embodiments, a handle may comprise two or more pieces; in some embodiments, a handle may comprise more than one material; in some embodiments, a handle may comprise a single piece and/or a single material. In some embodiments a handle may comprise an electronic device; in some embodiments, a handle may comprise chemical test strips. In some embodiments a handle may comprise a vial or other container for retrieving and/or collecting a volume of liquid.

In some embodiments, the means for securing a chemical test strip or other device to a handle may comprise a clip. Clips may comprise, but are not limited to, one or more of the following materials: ceramic, clay, cloth, crystal, fabric, fur and/or animal hide—e.g. leather, glass, metal and metal alloys (e.g. gold, silver, platinum, tin, aluminum, steel, stainless steel, iron, copper, and titanium), wood, rubber, other organic and/or synthetic polymer, Velcro, plastic (e.g. ABS, poly lactic acid), silicone, stone, and other natural and composite materials (e.g. carbon fiber).

Although it is not believed that drawings are necessary for the understanding of the subject matter sought to be patented, for illustrative purposes we have included several figures related to specific examples of embodiments of the disclosed invention.

In some of the figures and photographs of prototypes of some embodiments of the invention, clips are shown as a single printed plastic piece, although clips according to various embodiments of the instant invention may be formed, molded, cast, printed, carved, etched, milled, poured, or otherwise fabricated in more than one piece and/or assembled. In some embodiments, all or part of a clip may be conformal, flexible, malleable, moveable, pliable, plastic, rubbery, and/or stretchable.

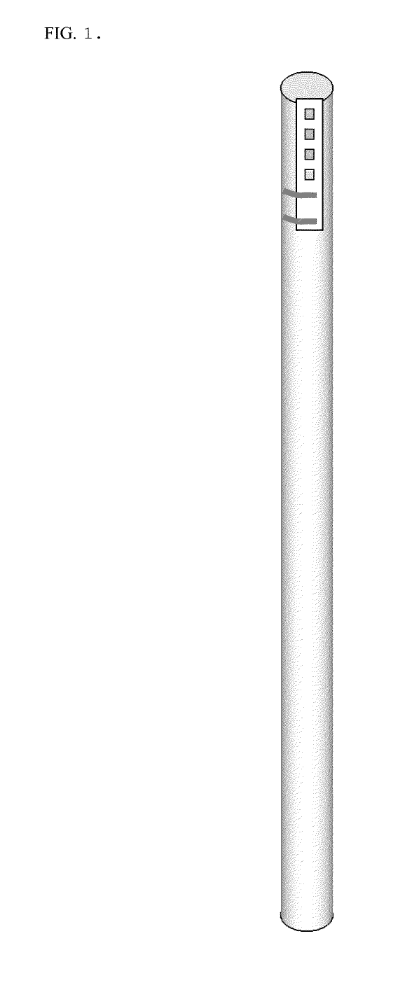

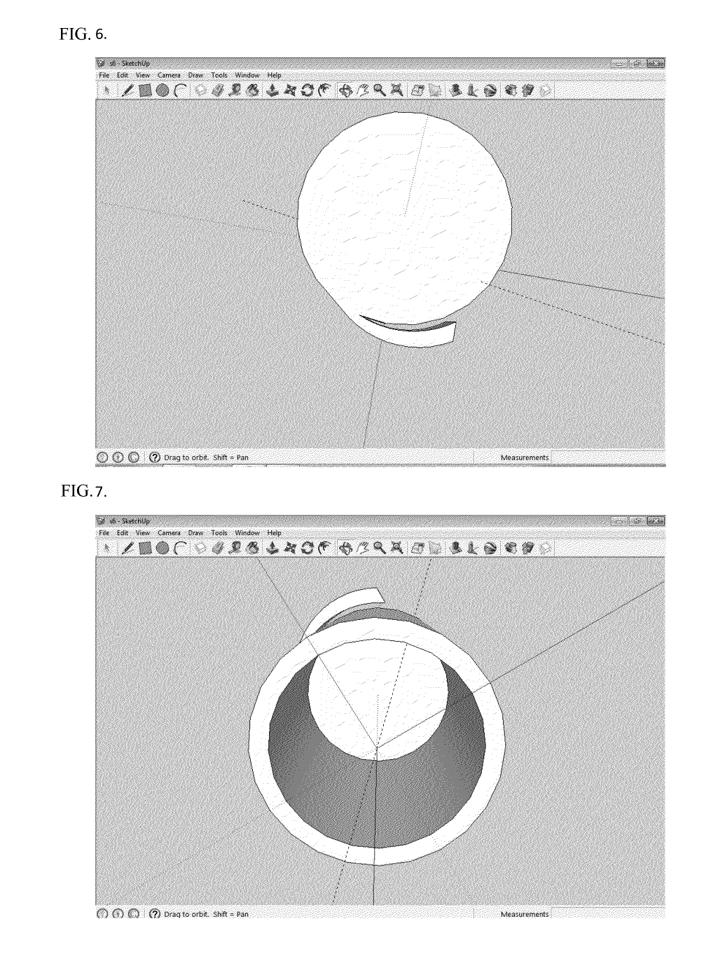

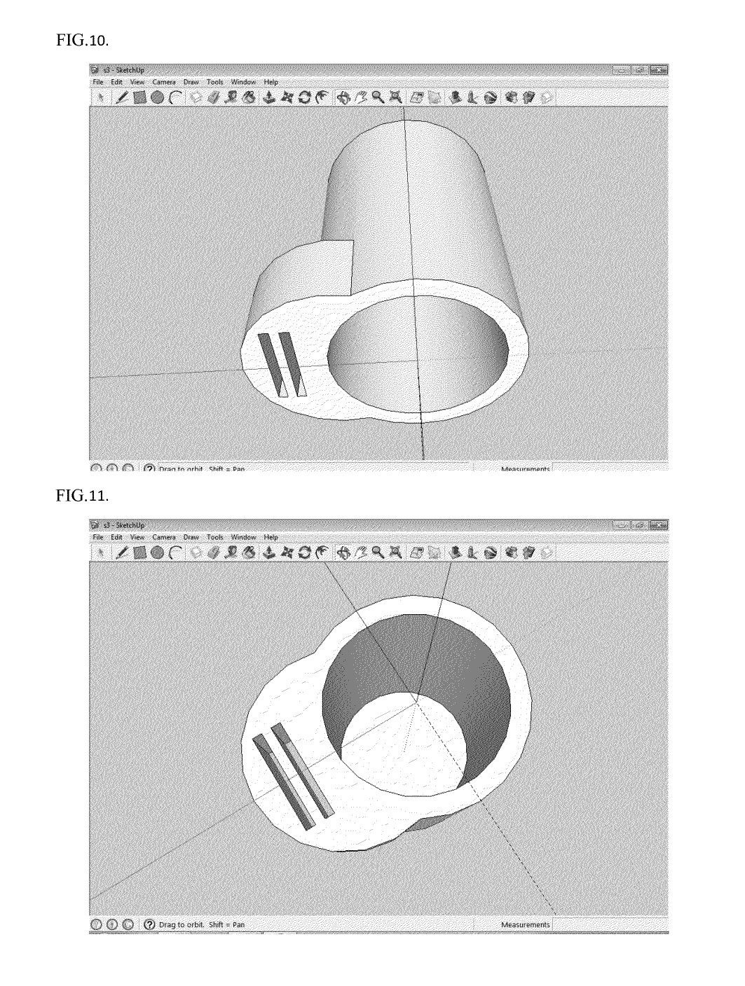

FIGS. 1, 4-11, and 15-18 illustrate specific examples of some embodiments of the instant invention comprising clips. In some embodiments of the invention, clips secure chemical test strips by compressing a portion or all of the test strips between one or more surfaces of the clips. As one specific example of an embodiment of the invention, FIG. 1 schematically shows a double-pronged clip attached to a handle so that the distance or gap between the inner surface of the clip prongs and the handle is smaller, in one or more regions, than the thickness of a test strip. In this specific example, when the prongs of the clip are disposed over a test strip (or when a test strip is inserted between the inner surfaces of the prongs of the clip and the handle), regions of the inner surfaces of the clip experience a force from the test strip pressing against them and this pressure torsions a portion of the clip connecting the surfaces, which exerts a proportional compressive pressure or force against the test strip and handle to hold them securely in place.

In some embodiments, clips may secure test strips in whole or in part by frictive forces. FIGS. 1, 4-11 and 18 illustrate embodiments where test strips are secured by clips in whole or in part by frictive forces. In some embodiments, clips secure testing and/or collection devices including but not limited to test strips in conjunction with bands and/or pins; in these instances bands and/or pins may provide additional compressive and/or frictive and/or other forces to keep testing and/or collection devices secure.

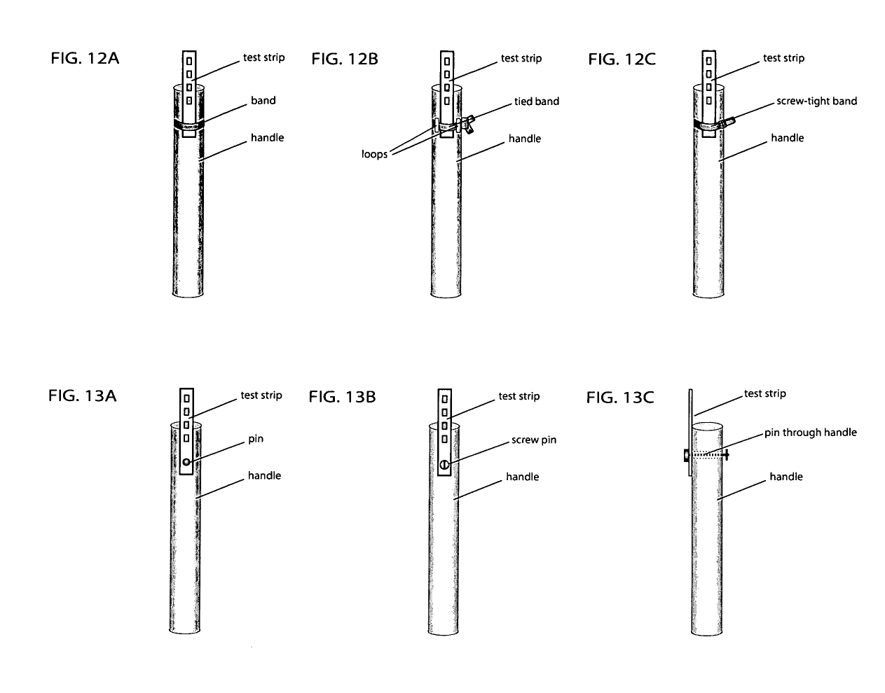

In some embodiments of the invention, the means for securing testing and/or collection devices including but not limited to test strips comprises one or more bands. FIGS. 12 A-C illustrate specific examples of some embodiments of the instant invention comprising bands. In some embodiments, bands comprise, but are not limited to, one or more of the following materials: ceramic, clay, cloth, crystal, fabric, fur and/or animal hide—e.g. leather, glass, metal and metal alloys (e.g. gold, silver, platinum, tin, aluminum, steel, stainless steel, iron, copper, and titanium), wood, rubber, other organic and/or synthetic polymer, Velcro, and plastic, silicone, stone, and other natural and composite materials (e.g. carbon fiber). In some embodiments, bands comprise a single unbroken loop of material. In some of the figures and photographs of prototypes of some embodiments of the invention, bands are shown as a single strip of fabric, although bands according to various embodiments of the instant invention may be formed, molded, cast, printed, carved, etched, milled, poured, or otherwise fabricated in more than one piece and/or assembled. In some embodiments, all or part of a band may be conformal, flexible, malleable, moveable, pliable, plastic, rubbery, and/or stretchable. In some embodiments, bands may be adjustable to permit the band to encircle and/or secure a larger or smaller area. In some embodiments, bands may comprise one or more pieces of material, which may be joined with or without fasteners. In some embodiments, fasteners may comprise, but are not limited to, adhesives, buckles, buttons, clasps, clips, slides, snaps, ties and Velcro. In some embodiments, bands may comprise strings, ropes, woven, stitched, embroidered, or other assembled materials. In some embodiments, bands may additionally comprise compartments, enclosures, holders, and pouches. In some embodiments, bands may be coated in whole or in part with adhesives comprising, but not limited to, contact adhesives, drying adhesives such as Elmer's™ glue, epoxies, hot melt and/or heat activated adhesives, polymer adhesives such polyvinyl acetate, pressure sensitive adhesives, rubber cement, silicone based adhesives, and other natural and synthetic adhesives.

In some embodiments of the instant invention, the means for securing testing and/or collection devices including but not limited to test strips comprises one or more pins. Some embodiments of pins comprise, but are not limited to, one or more of the following materials: ceramic, clay, cloth, crystal, fabric, fur and/or animal hide—e.g. leather, glass, metal and metal alloys (e.g. gold, silver, platinum, tin, aluminum, steel, stainless steel, iron, copper, and titanium), wood, rubber, other organic and/or synthetic polymer, Velcro, and plastic, silicone, stone, and other natural and composite materials (e.g. carbon fiber). FIGS. 13 A-C illustrate specific examples of some embodiments of the instant invention comprising pins.

Some embodiments of the instant invention comprise combinations of one or more of a clip, a band, and a pin.

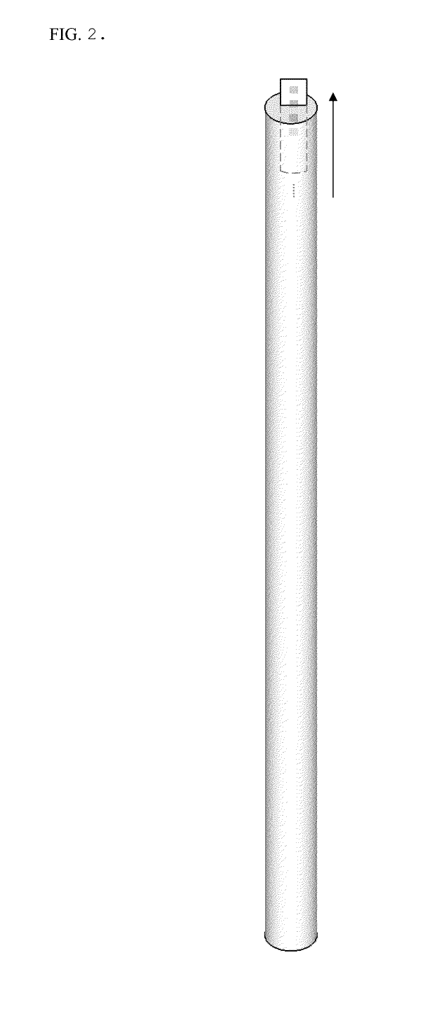

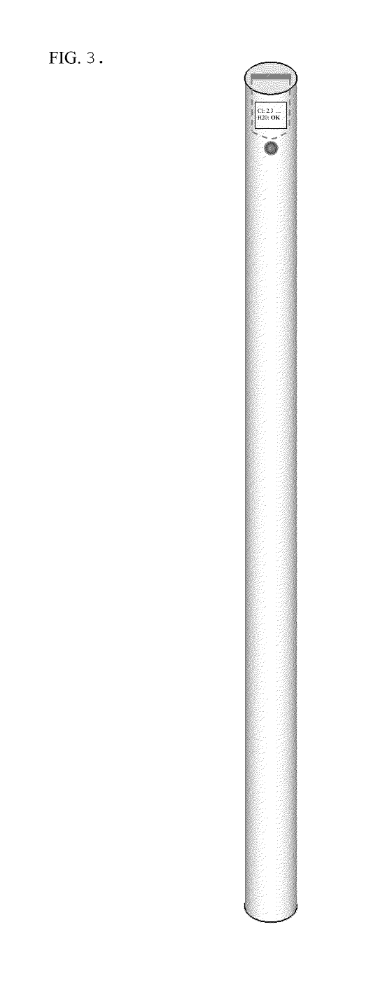

FIG. 1 illustrates one specific example of an embodiment of the instant invention comprising a two-pronged clip securing a test strip to a handle. FIG. 2 illustrates another specific example of an embodiment of the instant invention comprising a handle with a roll of test strips inside a portion of the handle, one of said test strips being exposed through a slit in an end of said handle. FIG. 3 illustrates one specific example of an embodiment of the instant invention comprising an electric test device embedded in an end of a handle; in this specific example, the OLED screen displays salient data and the green light indicates water quality is within desired parameters.

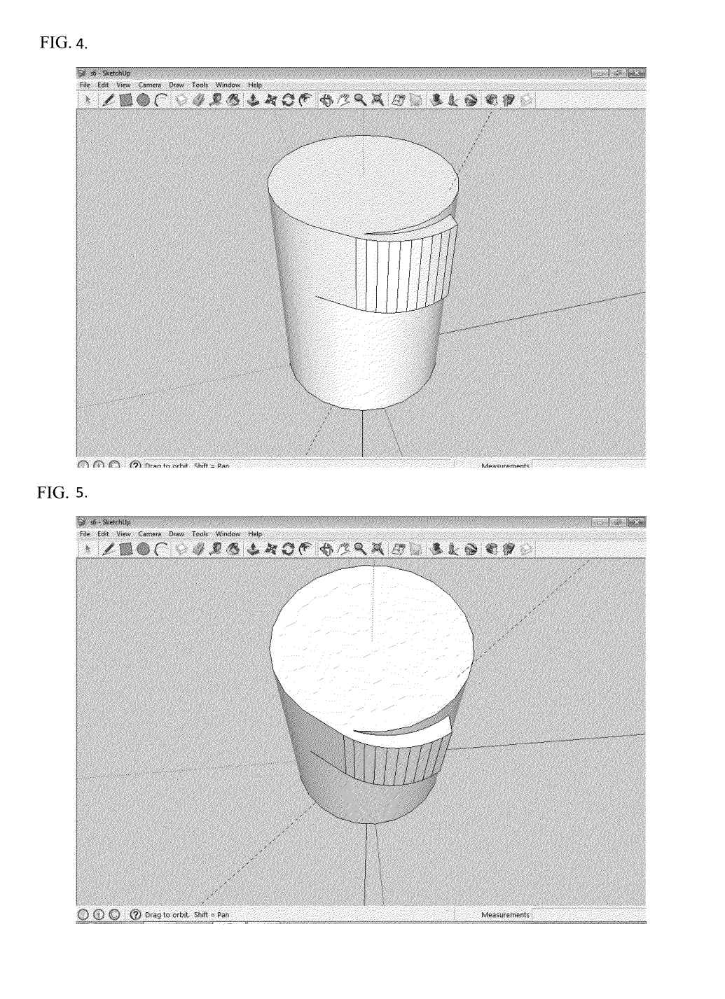

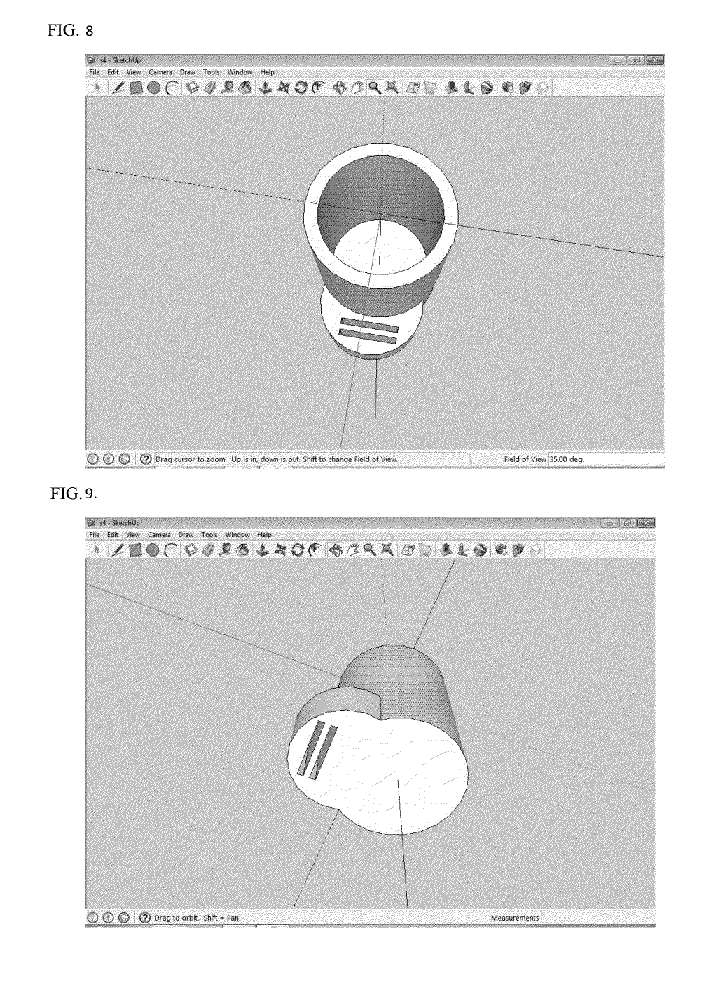

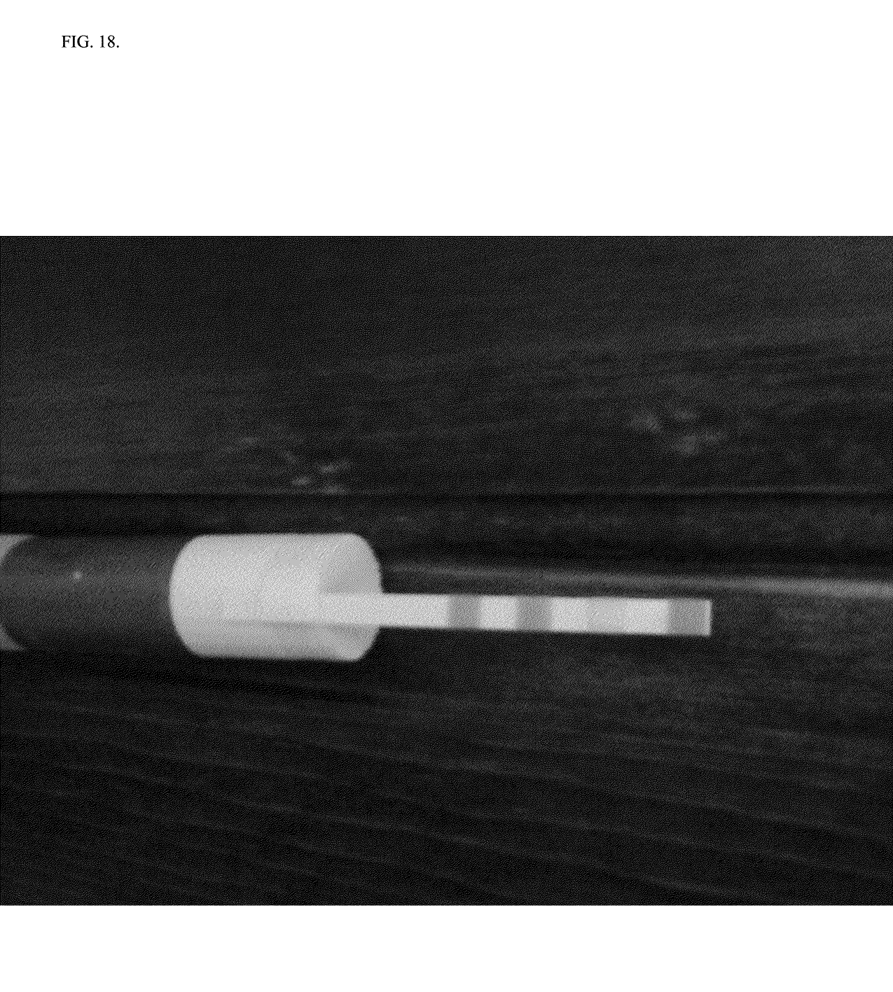

FIGS. 4-11 are 3D schematics of several specific examples of embodiments of the instant invention comprising clips. FIGS. 4-7 show different views of an example of an embodiment of a clip that may be detachably affixed to a handle and which is used to secure a test strip as shown in FIG. 18. FIGS. 8-11 illustrate different views of an example of an embodiment of a clip that may be detachably affixed to a handle and which may be used to secure a test strip by inserting one end of the strip through one slot and bending it back to insert the opposite direction through the other slot.

FIGS. 12 A-C illustrate specific examples of embodiments of the instant invention comprising bands—respectively elastic, tied, and screw-tied. FIGS. 13 A-C C illustrate specific examples of embodiments of the instant invention comprising pins—respectively straight, screw-pin, and through-hole-pin.

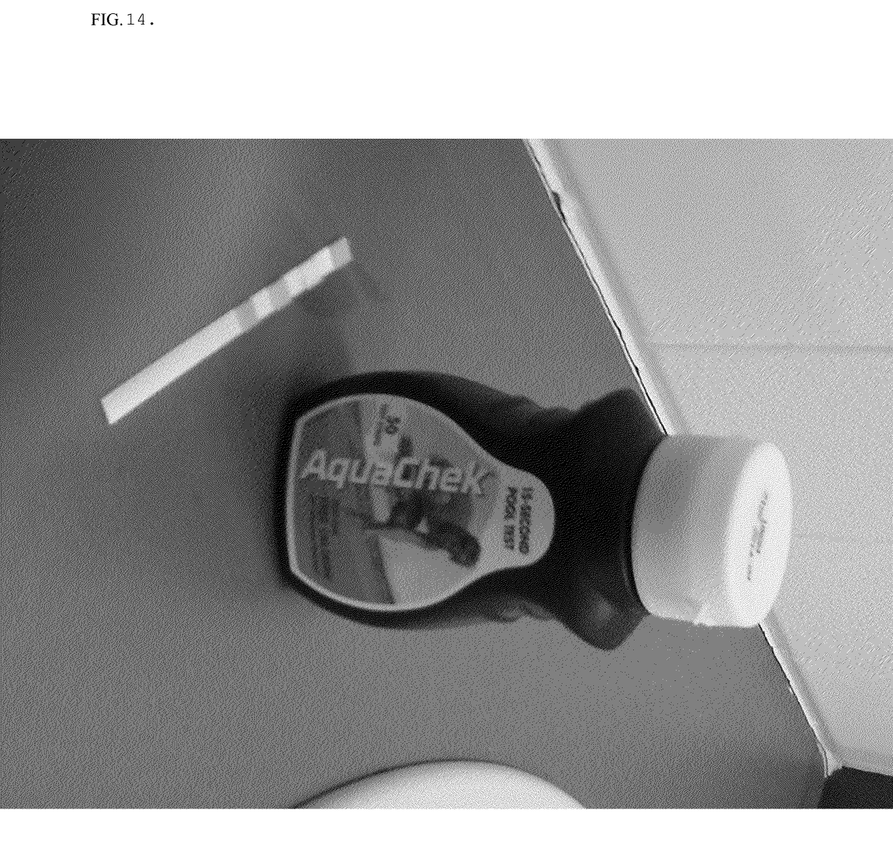

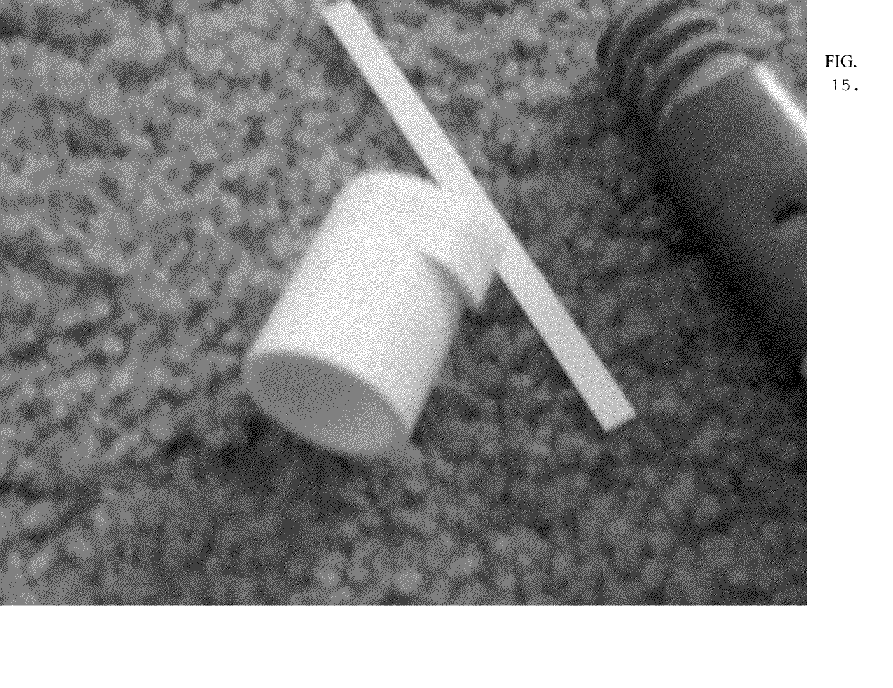

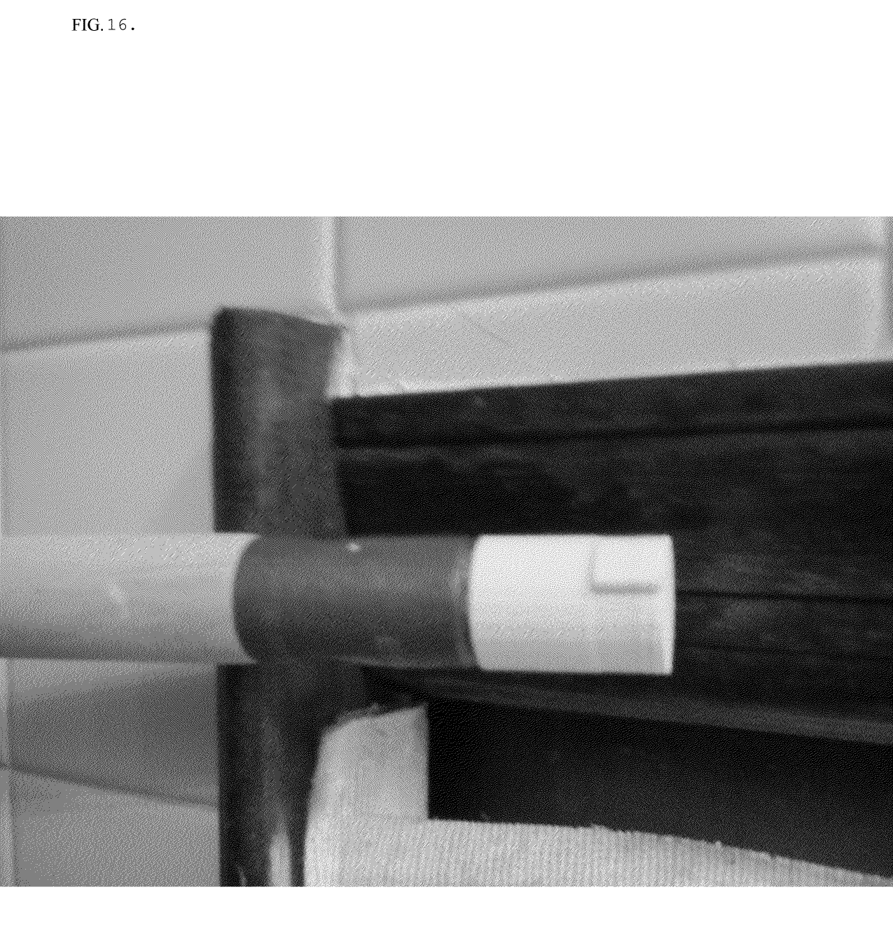

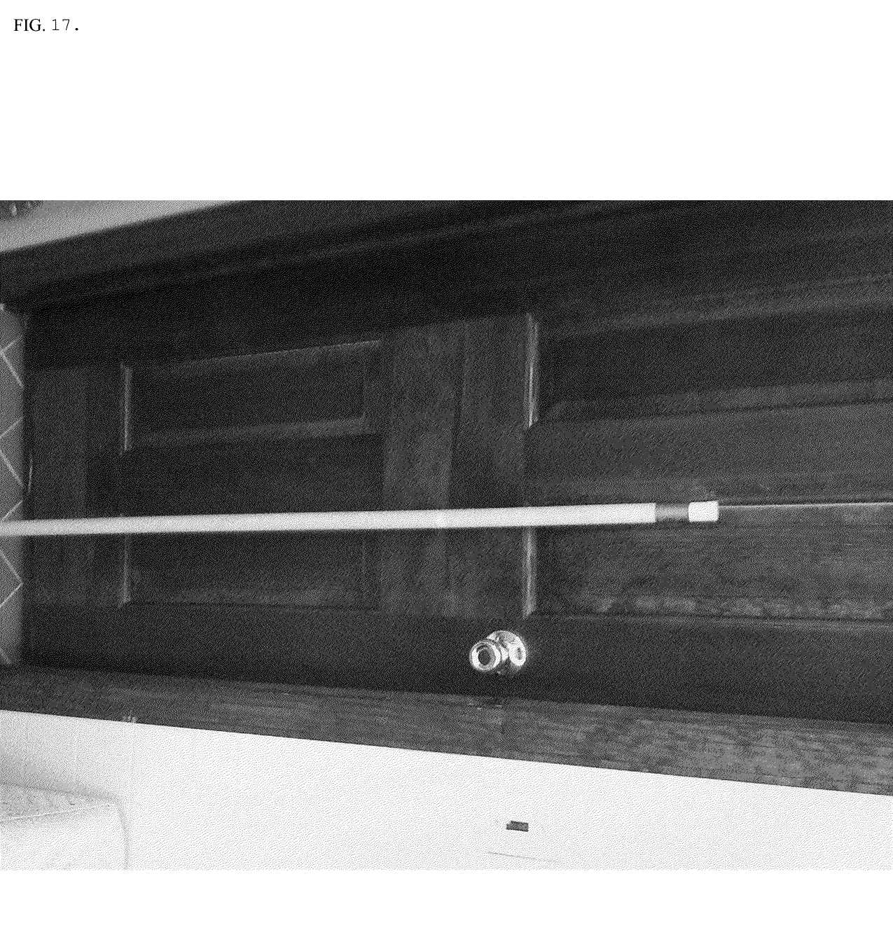

FIG. 14 is a photograph of a bottle of one brand of chemical test strips and a single unused test strip alongside it. FIG. 15 is a photograph of a specific example of an embodiment of a clip alongside the tip of a handle and a test strip. FIGS. 16 and 17 are a pair of photographs of a specific example of an embodiment of a clip affixed to a handle. FIG. 18 is a photograph of a specific example of an embodiment of a clip affixed to a handle with a test strip secured by the clip.

In some embodiments of the instant invention clips, bands, and pins may additionally comprise electronics such as lights, microphones, speakers, sensors and integrated circuits for interacting with users and/or other devices. For example, in some embodiments, lights and speakers may be added; adding sound and light may enhance visibility, safety and ease of use. As one specific example of a prototype of an embodiment of the invention, battery-powered LED lights have been added to one end of some handles to aid in attaching test strips and visualizing the depth of submersion; as another specific example, speakers may be used to recite directions (e.g., “deeper” “time's up”) or to generate other sounds such as a timer buzzer; as another specific example, microphones may be used to record sound for playback and to listen for voice commands; as another specific example, accelerometers may be embedded in clips, bands, pins, handles and may additionally provide feedback to a user.

In some embodiments of the invention, safety measures may be incorporated without departing from the spirit of the invention.

Some embodiments of the invention may additionally comprise sensing means, including but not limited to, one or more of the following: acceleration and/or rotation sensing means, impedance sensing means, capacitive sensing means, optical sensing means, pressure sensing means, force sensing means, other electromagnetic sensing means, other electromechanical sensing means, position sensing means, proximity sensing means, ultrasonic sensing means, and chemical sensing. In some embodiments, optical sensors including but not limited to active pixel sensors, CCDs, photodiodes, phototransistors, and custom or commercial-off-the-shelf (COTS) imagers, may be used to sense the environment. For some embodiments, chemical sensors may be used to sense the environment. Examples of commercial sensors of include but are not limited to those which may be found on the DigiKey website, www.digikey.com, the Allied Electronics website, www.alliedelec.com, and the website www.globalspec.com, and details pertaining to these sensors may be found in their associated sensor and product data sheets and published specifications.

Some embodiments of the invention may additionally comprise other hardware for performing other functions, including but not limited to communicating and/or interfacing with other circuits, components, devices, systems, networks and individuals, driving one or more display elements, processing and transmitting data, processing and transmitting power, storing and retrieving data, as well as for such miscellaneous functions as, e.g. waking and sleeping. Some embodiments may include power supplies; illustrative examples of power supplies include, but are not limited to, a battery holder, solar cells and associated charging circuitry, a plug for receiving wall power with or without associated circuitry (such as electrical transformers, rectifiers, voltage regulators, capacitors, etc.), an inductive power receiver element (such as a coil and circuitry to receive inductively coupled power), an electromechanical generator (think self-winding watch), a thermal generator (think MEMS/NEMS generators), and any other suitable source of power. Some embodiments also contain a communications module for transmitting and receiving data, including the position and/or location of other entities or markers, firmware and software updates, as well as other data (e.g., the time and date, weather conditions, etc. . . . ). A communications module may communicate wirelessly along one or more wavelengths of the electromagnetic spectrum, including but not limited to, radio waves, IR, and visible light (e.g. via radio transceiver, IR transceiver, other coded pulsed light transmissions, etc. . . . ). It may also communicate via wires which are not illustrated, for example using one of a variety of USB cables. In addition, a communications module may include speakers and/or microphones and associated circuits for receiving and decoding voice commands and for generating sounds, including but not limited to speech. It may also be integrated with a power supply—for example by capturing, harvesting and/or storing ambient or transmitted energy from an electromagnetic (“EM”) signal. Displays may comprise, but are not limited to the following: liquid crystals, thin film transistors, incandescent lights, fluorescent lights, light emitting diodes, organic light emitting diodes, lasers, fiber optics, color-changing polymers, pigmented fluids, solutions and mixtures, functionalized micro-beads, and e-inks.

In some embodiments of the instant invention, bands, clips, pins, handles, including but not limited to included hardware and/or software, may be manufactured, fabricated, selected, and/or assembled so as to be capable of withstanding prolonged exposure to high and/or low temperatures, immersion in fluids, and/or other extreme conditions. As one specific example, some embodiments of the instant invention are fabricated to be safely submersed in heavily chlorinated water without significant damage to the embodiment or degradation in performance.

Some embodiments of the invention may operate in conjunction with external devices, hardware, software, systems, and/or networks. For example, electronics such as a communications module inside a clip according to some embodiments of the instant invention may operate in conjunction with and be capable of transmitting data to and/or receiving data from an external device, system, or network using wires and/or wireless transmission methods. Illustrative examples of external devices comprise, but are not limited to, cell phones, tablet computers, PDAs, e-readers (e.g. Kindle™ and Nook™), hearing aids, laptop and desktop computers, monitors and displays, televisions, calculators, iPods™ and MP3 players, radios and stereos, watches with electrical circuits, remote controls, bar code readers, keyboards, cameras, other input devices, data acquisition systems, other electrical devices comprising, e.g., microcontrollers, programmable interface controllers, digital signal processors, memories, field programmable gate arrays, discrete circuits, and other electrical circuits and hardware, including custom application specific integrated circuits (ASIC). In addition, the world-wide-web, the Verizon™ wireless 4G LTE™ cellular network, and LANs are three illustrative examples of networks.

It should be noted that the figures and examples they represent are provided for illustrative purposes only and are not intended to limit the scope of the instant invention.

Although the foregoing invention has been described in some detail by way of illustration and example for purposes of clarity and understanding, it will be readily apparent to those of ordinary skill in the art in light of the teachings of this invention that certain changes and modifications may be made thereto without departing from the spirit and purview of this application or scope of the appended claims. All publications, patents, and patent applications cited herein are hereby incorporated by reference in their entirety.

I claim:

1. A means comprising one or more of a clip, a band and a pin for securing a chemical test strip or other test device to a handle.

2. The means of claim 1 wherein said handle comprises one or more of a clip, a hand and a pin.

3. A method for performing pool and other water and liquid testing comprising the steps of submersing all or a portion a testing apparatus into a region of a liquid for some period of time, and then removing all or a portion of the testing and apparatus from the liquid for evaluation.

4. The method of claim 3 wherein the testing apparatus comprises a chemical test strip and a handle having a means for securing the test strip to the handle.

5. The method of claim 4 wherein the method further comprises the steps of attaching the chemical test strip to the handle prior to submersing all or a portion of the testing apparatus into a region of a liquid.

6. The method of claim 3 wherein said testing apparatus comprises a handle comprising one or more chemical test strips.

7. The method of claim 6 wherein the method further comprises the steps of completely and/or partially dispensing one or more chemical test strips from the handle prior to submersing all or a portion of the handle comprising one or more chemical test strips into a region of a liquid for some period of time.

8. The method of claim 6 wherein the method further comprises the steps of submersing all or a portion of a handle comprising one or more chemical test strips into a region of a liquid then completely and/or partially exposing one or more regions of one or more chemical test strips to the liquid for some period of time.

9. The method of claim 6 wherein individual test strips may be unrolled, peeled off, or otherwise exposed to the environment to permit testing.

10. The method of claim 3 wherein said testing apparatus comprises an electronic hardware device.

11. The method of claim 10 further comprising the steps of submersing said device into a region of a liquid for some period of time.

12. The method of claim 11 further comprising the step of removing all or a portion of the device for evaluation.

13. The means of claim 1 wherein said means is detachable from said handle.

14. The means of claim 1 wherein said means is removable from said handle.

15. An apparatus comprising a handle.

16. The apparatus of claim 15 further comprising a clip.

17. The apparatus of claim 15 further comprising one or more electronic hardware devices.

18. The apparatus of claim 17 wherein one or more of said devices comprises lights.

19. The apparatus of claim 17 wherein one or more of said devices comprises a sensor.

20. The apparatus of claim 17 wherein one or more of said devices comprises a wireless communication apparatus.
